(19) United States
(12) Patent Application Publication
(10) Pub. No.: US 2026/0200991 A1
(43) Pub. Date: Jul. 16, 2026

(54) ENGINEERED VIRUS-LIKE-PARTICLES FOR TARGETED CAPTURE OF MEMBRANE PROTEINS

(71) Applicants: Mart Ustav, JR., Tartu (EE); Sachdev Sidhu, Toronto (CA)

(72) Inventors: Mart Ustav, JR., Tartu (EE); Sachdev Sidhu, Toronto (CA)

(21) Appl. No.: 19/138,302

(22) PCT Filed: Dec. 14, 2023
(86) PCT No.: PCT/IB2023/062716

§ 371 Date: Jun. 12, 2025

Related U.S. Application Data
(60) Provisional application No. 63/387,302, filed on Dec. 14, 2022.

(51) Int. Cl. C07K 14/16; A61K 38/00; A61K 39/00; A61K 39/21; C07K 14/705; C07K 16/00; C12N 7/00; G01N 33/53

(52) U.S. Cl. CPC C07K 14/161; A61K 38/00; A61K 39/21; C07K 14/705; C07K 16/005; C12N 7/00; G01N 33/53; A61K 2039/5258; A61K 2039/543; C07K 2319/02; C07K 2319/735; C12N 2740/16023; C12N 2760/14123

(57) ABSTRACT

Disclosed are engineered virus-like-particles (VLPs) and their use. In the VPLs integral plasma membrane proteins are captured and displayed on the surface of a VLP in a native conformation based on an interaction between a viral scaffolding protein fused to a PDZ domain and a synthetic polypeptide in the cytoplasmatic C-terminal tail of the target membrane protein.

### SEQUENCE LISTING

The sequence listing entitled as 11641-002WO1.XML, created on Dec. 13, 2023, and having a file size of 8,104 bytes is hereby incorporated by reference pursuant to 37 C.F.R. section 1.52 (e) (5).

### FIELD OF THE INVENTION

The invention relates to engineered virus-like-particles (VLPs) in which integral plasma membrane proteins are captured and displayed on the surface of a VLP in a native conformation based on an interaction between a viral scaffolding protein fused to a PDZ domain and a synthetic polypeptide in the cytoplasmatic C-terminal tail of the target membrane protein. The invention further relates but is not limited to the use of such engineered pseudotyped VLPs for antibody discovery.

### BACKGROUND OF THE INVENTION

Modulating biologic molecules that target membrane proteins have become one of the most promising therapeutics class (1). Antibodies as drug candidates have several advantages over small molecules, including better specificity, lower dosing frequency, and restriction from the central nervous system. There are two fundamental approaches for antibody development: the utilization of animal immune systems, or synthetic molecular display methods (e.g., those based on phage display) (2).

To harness the immune systems of various organisms for raising antibodies, the target antigen is presented to be recognized by the host humoral immune system, resulting in the activation of B cells and secretion of antibodies that recognize the presented epitopes of the antigen. Next, individual monoclonal antibodies can be isolated and produced through recombinant or hybridoma technologies.

Phage display of synthetic antibody fragments has been proven to be an effective molecular display tool for antibody developments that allows for the selection of antibodies against a vast array of biological and non-biological targets and provides an opportunity for linking the antibody phenotype and genotype (3) (4).

Furthermore, the recombinant in vitro nature of phage display gives way to using alternative depletion steps, epitope steering, binding to protein-protein interaction complexes, and the use of antigens of complex composition for binding.

G-protein coupled receptors (GPCRs) are one of the largest protein superfamilies with more than 800 members (5). Ion channel proteins are the second largest membrane protein class that is an important class of multi-transmembrane proteins expressed in almost all living cells and are involved in regulating a variety of physiological processes (6). Aberrant signalling of GPCRs and ion channel proteins has been linked to numerous immunological, neurological, and metabolic disorders, as well as cancers.

About 35% of all approved drugs target GPCRs, making them the largest family of proteins targeted for drug development thus far (7). However, in terms of antibody development strategies, multi-transmembrane proteins are a challenging group of targets. They are frequently expressed at low density on the cell surface and are unstable and misfolded when removed from the lipid bilayer. As a result, obtaining sufficient amounts of relevant antigens for antibody discovery efforts by either antibody display techniques or through immunizations of animals has remained one of the limiting factors, thus making the development of specific antibodies very challenging.

Furthermore, the structure of GPCR and ion channel targets itself enfolds another limiting factor, with minimal epitopes displayed on the cell surface for potential binding of antibodies.

To date, most anti-GPCR antibodies have been raised against the larger N-terminal extracellular domains or linear peptides with GPCRs. Producing appropriate full-length GPCR antigens has remained the largest bottleneck (8).

As developing immune derived antibody libraries against evolutionally conserved membrane proteins has proven to be difficult, the use of phage displayed synthetic antibody fragments has been an effective molecular tool for antibody development that allows for the selection of antibodies against a vast array of biological and non-biological targets (9).

Formed by assembled capsid proteins that are surrounded by cell membrane, virus-like particles (VLPs) represent a suitable model for the expression of membrane-bound proteins (10) (11). VLPs that are replication incompetent macromolecular protein assemblies formed by a minimum of a single viral protein have been developed for a number of different viruses (12). By co-overexpressing a target membrane protein in a host cell, it is possible to generate pseudotyped VLPs (11). At the same time, using VLPs as a “snapshot” tool for capturing the high transiently overexpressing cell membrane state enables these particles to be used as antigens for selections in molecular display technologies and have been used successfully for development of antibodies against multi-transmembrane proteins for which traditional recombinant expression systems are not feasible (13).

There is a continuing need for methods to develop novel antibodies, as well as a need to provide novel systems that would allow high yield of full-length membrane proteins for purpose of developing and screening novel antibodies for novel drugs and vaccines. Furthermore, there is a need for methods to provide VLPs for providing efficient immune response, for developing new vaccines and treatments.

### SUMMARY OF THE INVENTION

This invention is aimed to provide solutions to the above-described problems and more. The invention provides constructs and methods to enhance capture and display of membrane proteins, preferably integral membrane proteins, especially, G-protein coupled receptors (GPCRs), ion channels, tetraspanins and single-pass membrane proteins, however not limited to these groups.

This invention provides a membrane protein expression system based on virus-like particles (VLP) enabling expression/capturing membrane proteins on the surface of VLPs in a significantly more enhanced level than currently available methods.

Inspired by the fact that several viral capsid scaffolding proteins interact intracellularly with the cytoplasmatic tails of viral envelope glycoproteins in order to effectively incorporate them on the surface of the VLP (14) (15), we developed a HIV-1 gag and Ebola VP40 based membrane protein expression system where the respective viral scaffolding gag and VP40 proteins would interact with the cytoplasmatic tail of a target membrane protein, thereby facilitating an increased capture and display of the target membrane protein.

To achieve this we fused the viral gag and VP40 proteins with a Erbin PDZ domain that is known to interact tightly and specifically to a 7 amino acid synthetic peptide (16). Ebola VP40-PDZ amino acid sequence is shown as SEQ ID NO:1; HIV-1GAG-PDZ is shown as SEQ ID NO: 2. Erbin PDZ domain is SEQ ID NO:3. PDZ domains are known to interact with short C-terminal amino acid motifs and thereby are suitable for developing such interactions. In addition, PDZ domains are often shown to interact with C-terminal tails of GPCRs and play a role in the transport and recycling of GPCRs to the plasma membrane (17). The ability of PDZ domains to promote recycling and stabilize interacting membrane proteins further supported the rationale of utilizing such an interaction (18). More specifically, Erbin has been shown to interact and stabilize membrane-bound Erbb2 (19).

We surprisingly found that based on the above-described system, capture and display of class A, B, and F GPCRs, ion channels, tetraspanins, and single-pass membrane proteins can be increased remarkably. The system according to this disclosure can provide expression of GPCRs, ion channels, tetraspanins and single-pass membrane proteins in 3 to 5 times higher levels compared to using wild-type VLP expression constructs.

In summary we report the development of a robust membrane protein expression system based on an interaction between the viral scaffolding proteins and target membrane protein cytoplasmatic protein motifs that enable a high-density display of membrane proteins that can be used for the development of a variety of assay systems and, more importantly, represents an efficient antigen system for antibody development against therapeutically relevant membrane protein classes.

Accordingly, it is an object of this invention to provide a virus-like-particle (VLP) expressing one or more membrane embedded proteins on its surface and comprising a viral scaffolding protein fused to a PDZ domain and a synthetic polypeptide in cytoplasmic C-terminal tail of the one or more target membrane proteins, and optionally a signal peptide incorporated at the N-terminal end of the one or more target membrane proteins.

In one aspect of the invention the optional signal peptide incorporated at the N-terminal end of the target membrane protein may be a signal peptide of Albumin, Melittin, Pr-MCH, Gaussia luciferase, Heamagluttinin, HIV-Glycoproitein, LRRCP32, Growth Hormone Receptor, Proteinase activated R1, Tissue type Plasminogen activator, Secrecon, AcMNPVEnv, Somatrotopin, IL-2, MCHR-1, but not limited to these.

According to certain aspects of the invention the PDZ domain fused to the scaffolding protein is according to SEQ ID NO:3.

According to certain aspects of the invention the viral scaffolding protein can be a member of the Filoviridae family, e.g. Ebola virus VP40, or Retroviridae family member of HIV-1or MLV virus gag protein, however, not limited to these.

According to certain aspects of the invention, the VLPs express one or more membrane proteins selected from the group consisting of G-protein coupled receptors (GPCRs), ion channels, tetraspanins and single-pass membrane proteins.

According to certain aspects of the invention, in the VLPs the synthetic polypeptide fused to the pseudotyped membrane protein is according to SEQ ID NO:6.

It is another object of this invention to provide a VLP-vaccine or a VLP-preparation consisting of or comprising VLPs expressing one or more membrane embedded proteins on its surface and comprising a viral scaffolding protein fused to a PDZ domain and a synthetic polypeptide in cytoplasmic C-terminal tail of the one or more target membrane proteins, and optionally a signal peptide incorporated at the N-terminal end of the one or more target membrane proteins.

According to certain aspects of the invention the VLP-vaccine or -preparation is injectable, an intranasal or an inhalable.

It is yet another object of the invention to provide a method to generate an immune response, the method comprising administering to an animal a vaccine or a preparation comprising or consisting of VLPs expressing one or more membrane embedded proteins on its surface and comprising a viral scaffolding protein fused to a PDZ domain and a synthetic polypeptide in cytoplasmic C-terminal tail of the one or more target membrane proteins, and optionally a signal peptide incorporated at the N-terminal end of the one or more target membrane proteins.

According to certain aspects the invention includes a method to develop antibodies or antibody preparations or membrane protein binding affinity molecules through immunization of animals by administering to the animal a vaccine or preparation comprising or consisting of VLPs expressing one or more membrane embedded proteins on its surface and comprising a viral scaffolding protein fused to a PDZ domain and a synthetic polypeptide in cytoplasmic C-terminal tail of the one or more target membrane proteins, and optionally a signal peptide incorporated at the N-terminal end of the one or more target membrane proteins.

According to certain aspects the invention includes the antibodies obtained from an animal immunized by administering to the animal a vaccine or preparation comprising or consisting of VLPs expressing one or more membrane embedded proteins on its surface and comprising a viral scaffolding protein fused to a PDZ domain and a synthetic polypeptide in cytoplasmic C-terminal tail of the one or more target membrane proteins, and optionally a signal peptide incorporated at the N-terminal end of the one or more target membrane proteins.

It is a further object of this invention to provide a method to develop monoclonal antibodies in a phage display system by using the VLPs expressing one or more membrane embedded proteins on its surface and comprising a viral scaffolding protein fused to a PDZ domain and a synthetic polypeptide in cytoplasmic C-terminal tail of the one or more target membrane proteins, and optionally a signal peptide incorporated at the N-terminal end of the one or more target membrane proteins.

A further object of the invention are the antibodies obtained in a phage display system by using the VLPs expressing one or more membrane embedded proteins on its surface and comprising a viral scaffolding protein fused to a PDZ domain and a synthetic polypeptide in cytoplasmic C-terminal tail of the one or more target membrane proteins, and optionally a signal peptide incorporated at the N-terminal end of the one or more target membrane proteins.

Yet another object of the invention is to provide antibodies for disease treatment, and/or drug discovery.

A further object of the invention is to provide a membrane protein expression system comprising virus-like particles expressing one or more membrane embedded proteins on its surface and comprising a viral scaffolding protein fused to a PDZ domain and a synthetic polypeptide in cytoplasmic C-terminal tail of the one or more target membrane proteins, and optionally a signal peptide incorporated at the N-terminal end of the one or more target membrane proteins, wherein the expression system enables expression and capturing of the membrane proteins on VLP surface in levels that are enhanced as compared to a system comprising wild-type VLP construct.

### BRIEF DESCRIPTION OF THE DRAWINGS

FIG. 1A-C: A Schematic representation of HIV-1 gag/Ebola VP40 viral particle formation. Viral gag and VP40 oligomerize under the plasma membrane to form viral particles that bud out from the cell and capturing the host cell plasma membrane as an envelope. B Schematic flow-chart of the expression and purification of HIV-1 gag and Ebola VP40 derived VLPs. C Transmission electron microscopy images of HIV-1 gag and Ebola VP40 derived VLPs. HIV-1 gag VLPs compose of spherical particles with a diameter of 100-200 nm. Ebola VP40 forms filamentous particles with about 80-100 nm width but with variable lengths.

FIG. 2A: Schematic representation of the HIV-1 gag-Erbin PDZ fusion protein (SEQ ID NO: 2). The plasma membrane targeting matrix associated MA-domain is highlighted in orange. The CA domain responsible for gag oligomerization into particles is highlighted in blue. Then nucleocapsid domain (green) links the Erbin PDZ domain (SEQ ID NO:3) shown in blue. The TGWETWV binding peptide (SEQ ID NO:6) is shown in red.

FIG. 2B: Schematic representation of Ebola VP40-Erbin PDZ fusion protein (SEQ ID NO: 1). The Erbin PDZ domain (SEQ ID NO:3) shown in blue is fused to the N-terminus of VP40. The N-terminal domain of VP40 responsible for oligomerization and particle formation is shown in cyan. The C-terminal domain of VP40 responsible for plasma membrane targeting is shown in orange.

FIG. 3A-E: A Scheme of plasma membrane embedded GPCR with a synthetic intracellularly located C-terminal Erbin-PDZ domain binding peptide interacting with HIV-1 gag-Erbin PDZ protein. B The effect of heterologous signal peptides on the cell surface expression of a Class A GPCR MCHR1. Y-axis represents the relative median fluorescence intensity and X-axis represents the origin of the respective signal peptide sequence introduced to the N-terminus of MCHR1. C Normalized VLP based Flag-ELISA assay of HIV-1 gag VLPs expressed with various combinations that represent either no-interaction occurring between MCHR1 and viral scaffolding protein (PDZ-gag MCHR1 wt, gag MCHR1 wt, gag SP-MCHR1 wt) or variants where an interaction between MCHR1 and viral scaffolding gag molecule is occurring (PDZ-gag MCHR1 C-term pep (SEQ ID NO:4) and PDZ-gag SP-MCHR1 C-term pep SEQ ID NO:5)). A 5-fold increase in MCHR1 expression is detected with the PDZ-gag-MCHR1 interaction. Furthermore, the addition of a signal peptide to the N-terminus of MCHR1 has an additional synergistic effect on MCHR1-VLP expression levels based on detection of a N-terminal Flag tag of MCHR1. D The 7aa Erbin PDZ domain binding sequence was fused to the intracellular proximal C-terminus of Flag tagged Class F GPCRs Fzd5 and Fzd3, Class B GPCR GLP1R, ion channel P2RX3, single pass membrane proteins GFRAL and HIV-1 Env. The respective expression levels based on a Gag normalized Flag-ELISAs are demonstrated. An interaction specific increase in target membrane protein capture on VLPs is observed in all selected target protein except for HIV-1 Env protein. E The 7aa Erbin PDZ domain binding sequence was fused to the intracellular proximal C-terminus of Flag tagged Class F GPCRs Fzd5 and Fzd3, Class B GPCR GLP1R, ion channel P2RX3, single pass membrane proteins GFRAL and HIV-1 Env. The respective expression levels based on a VP40 normalized Flag-ELISAs are demonstrated. An interaction specific increase in target membrane protein capture on VLPs is observed in all selected target protein except for HIV-1 Env protein.

FIG. 4A: LC-MS/MS analysis of HIV-1 Gag-Erbin PDZ and Ebola VP40-Erbin PDZ derived VLPs pseudotyped with MCHR1. Venn diagrams indicating either the total proteome difference of HIV-1 and Ebola derived VLPs or the differences in VLP surface proteome. The column graph indicates number of total detected spectra per surface protein on the respective VLP.

FIG. 4B: Complete list of Ebola VP40-Erbin PDZ and HIV-1 gag-Erbin PDZ derived VLP specific and shared surfaceome proteins.

FIG. 5A-E: A Scheme of PDZ-VLP based phage displayed antibody selections. VLPs are coated on MaxiSorp solid surface plates and antibody Fab fragment displayed phage libraries are introduced to bind the VLPs overexpressing the target membrane protein. In each round the VLPs are alternated between HIV-1 and Ebola and a preclearing step against target negative VLPs is performed before introducing to the target positive VLPs. After 5 rounds of selections the target positive clonal phage are determined by phage-ELISA. B Phage-ELISA results of antibody selections performed against Fzd5-VLPs. Out of 180 clones analysed by Phage ELISA 175 were positive in binding to Fzd5-VLPs compared to target negative VLPs. A total of 44 unique antibody clones based on CDR H3 sequences were determined. C Specificity analysis of 7 Fzd5-VLP derived antibody in IgG1 format based on bio-layer-interferometry (BLI). D Fzd5 TopFlash assay to determine the functional ability of Fzd5-VLP derived anti-Fzd5 IgG to block canonical Wnt signalling. Luciferase signals are represented as relative to no antibody treated cells. E Cell proliferation assay of HPAF-II pancreatic cancer cell line in response to treatment to anti-Fzd5 13080, 13082 and isotype control 4275 antibodies. Cell viability was determined after treatment of cells with respective antibodies after 6 days using Alamar Blue assay.

FIG. 6: ELISA based membrane protein expression level comparison of different membrane proteins expressed on HIV-1 gag VLPs either with the EPEP peptide fused to the membrane protein C-terminus and expressed with a gag-ERBIN PDZ fusion protein based VLP or as a wt sequence. Throughout all examples the ERBIN-PDZ and EPEP interaction is providing enhanced display of the target membrane protein on the surface of the VLP.

### DETAILED DESCRIPTION OF THE INVENTION

### Definitions

By membrane protein it is meant here a protein that is attached to or associated with the membrane of a cell. A membrane protein may be an integral membrane protein penetrating through the cell membrane, or a peripheral membrane protein integrated on one side of the cell membrane, or a surface protein. A membrane protein may be a single transmembrane protein or a polytopic transmembrane protein. A membrane protein may be a transport protein. A membrane protein may be a carrier protein or a channel protein. A membrane protein may a receptor protein.

### Development of PDZ-VLP System

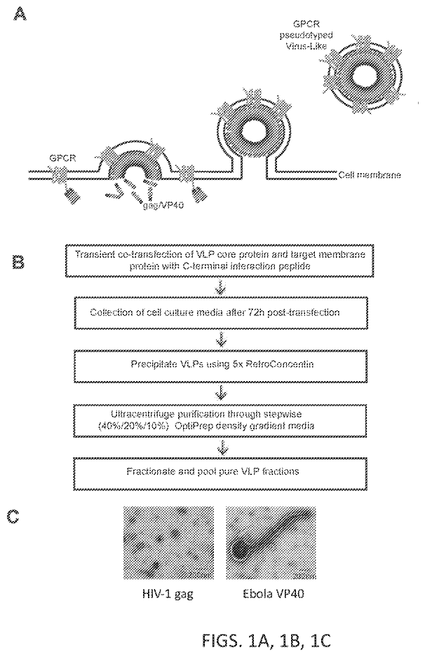

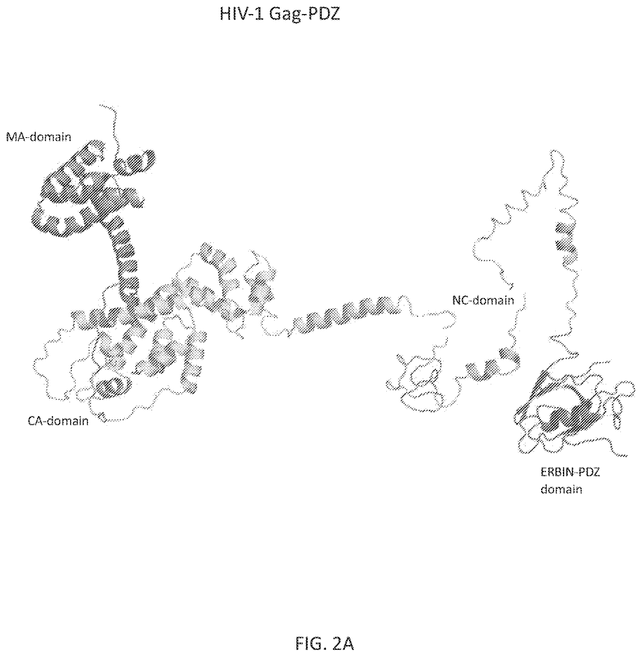

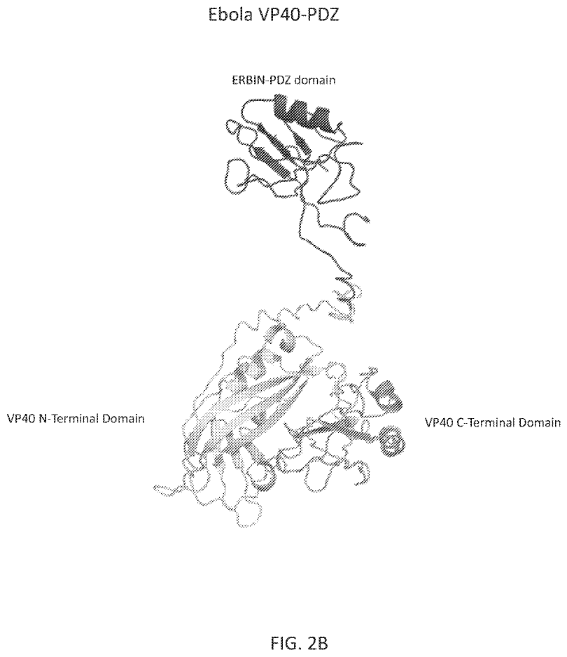

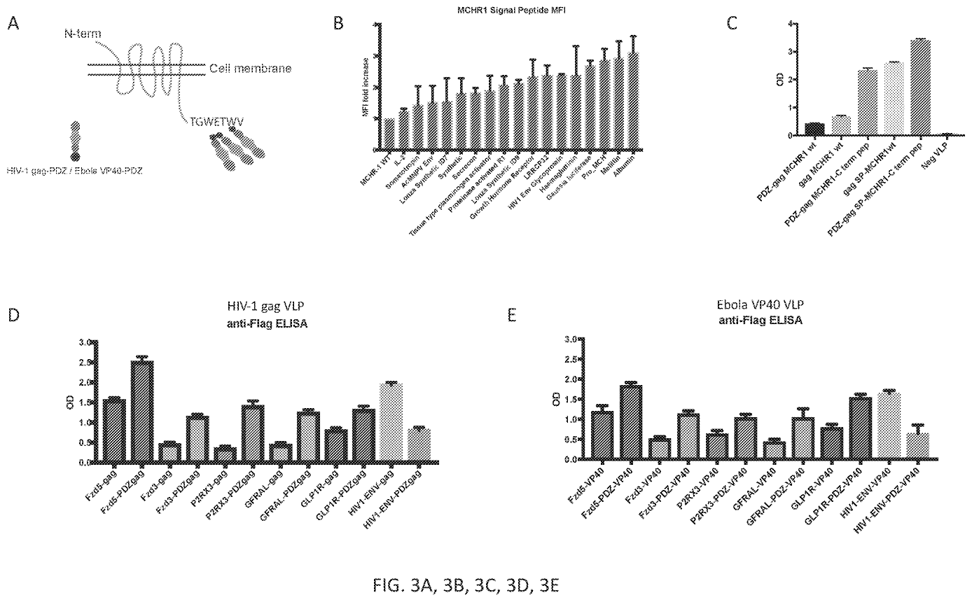

A key step for the efficient generation of modulating antibodies against membrane proteins is to retain and display the antigen epitopes in a relevant native context. Removing multi-transmembrane proteins like GPCRs and ion channels from the lipid bilayer can generate changes in the presented folds of the protein and yield in non-functional antibodies. The use of VLPs for capturing membrane proteins has been relatively well established, but with the efficiency determined by target protein expression levels and membrane microdomain localization in the host cells. The expression of retroviral HIV-1 gag protein and filoviral Ebola VP40 protein yields in the assembly and budding of VLPs from host cells (20) (21). The viral particles capture the host cell membrane as an envelope upon budding from the cell, thereby also enabling the capture of the transiently co-overexpressed membrane proteins. The development of efficient transient expression systems in serum-free conditions has enabled the development of a straightforward expression and purification pipeline by extracting the VLPs from expression supernatants (22) (FIG. 1). In order to further develop a robust system that would allow to efficiently capture and purify membrane proteins from different classes on VLPs, we engineered an interaction between the viral scaffolding proteins and the target integral membrane protein. As PDZ domains are known to interact with short C-terminal and internal peptide motifs and have been shown to stabilize and recycle membrane proteins on the plasma membrane, they represent a suitable functional domain for such engineering strategy. ERBIN is a member of the LAP (leucine-rich repeat and PDZ domain) protein family that has been shown to bind tightly to a synthetic phage display derived 7 amino acid peptide (16). We fused the ERBIN PDZ domain to the C-terminus of HIV-1 gag (FIG. 2a) and the N-terminus of Ebola VP40 (FIG. 2b). The 7aa TGWETWV ERBIN PDZ binding sequence is fused to the C-terminal tail of target membrane protein (FIG. 3A). Only about 10% of GPCRs are known to include a cleavable N-terminal signal peptide (23). As the incorporation of signal peptides to the N-terminus of membrane proteins has been shown to increase cell surface expression yields, we tested the effect of 17 different signal peptides sequences on a class A GPCR MCHR1 cell surface expression based on a flow cytometry analysis through a N-terminal Flag tag. As demonstrated on FIG. 3B, all of the introduced signal peptides resulted in increased cell surface expression of Flag-MCHR1 (SEQ ID NO: 4) with maximum of 3-fold increase in case of an albumin signal peptide.

In evaluating the effects of including a N-terminal signal peptide and C-terminal PDZ domain interaction tag on the pseudotyping level of MCHR1 on HIV-1 gag and gag-PDZ derived VLPs, we expressed and purified MCHR1 pseudotyped VLPs with different modifications and determined display levels through VLP-ELISA through a N-terminal Flag tag on MCHR1 (FIG. 3C). A 5-fold increase in MCHR1 expression was detected with the PDZ-gag-MCHR1 interaction based on normalized MCHR1-VLP Flag-ELISA. Furthermore, the addition of a signal peptide to the N-terminus of MCHR1 has an additional synergistic effect on MCHR1-VLP expression levels based on detection of a N-terminal Flag tag of MCHR1.

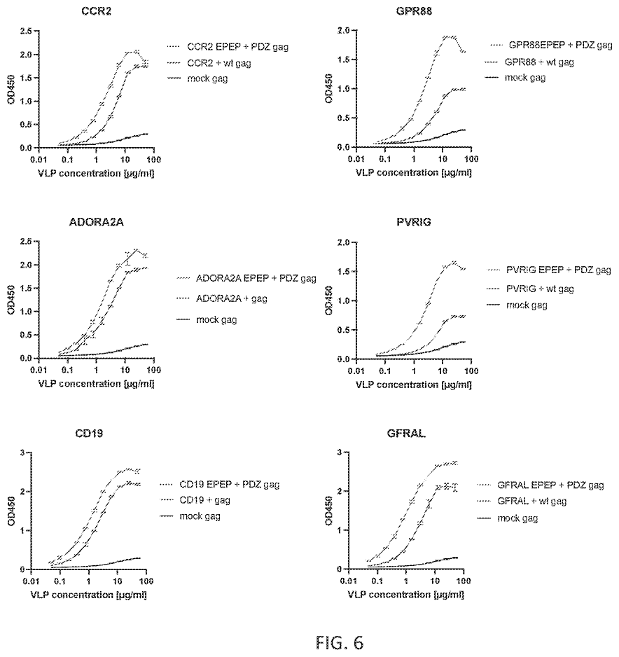

In order to furthermore demonstrate the robustness of this system to facilitate the capture of different membrane proteins, we expressed VLPs with overexpressed Class F GPCRs (Fzd5 and Fzd3), Class B GPCR (GLP1R), ion channel (P2RX3) and single pass membrane proteins (GFRAL and HIV-1 Env) (FIG. 3 D, E). When analysing the effects of the interaction for the incorporation of different membrane proteins on both HIV-1 and Ebola VP40 derived VLPs, we were able to see an increase of incorporation for all membrane proteins, except for HIV-1 Env protein, where the interaction had a negative effect, resulting in a 2-fold decrease of Env display compared with the Env-wt in a normalized VLP ELISA. In FIG. 6 we further demonstrate in a concentration dependent ELISA based assay the increased expression of target membrane proteins on the surface of VLPs due to the engineered interaction.

### Proteomic Analysis of Pseudotyped VLPs

Retroviruses and filoviruses are known to capture the host cell membrane as an envelope, thus also incorporating host proteins into viral particles. In addition to integral membrane proteins displayed on the surface of the particles, a number of cytoplasmic proteins are captured as interaction partners with intracellular domains of integral membrane proteins and the viral Gag and VP40 protein (11) (24). A study analysing the protein composition of HIV-1 cores expressed in different cell types has shown that only 42 out of 202 proteins could be detected in all VLP samples indicating a high degree of host cell dependency for the VLP proteome composition (25).

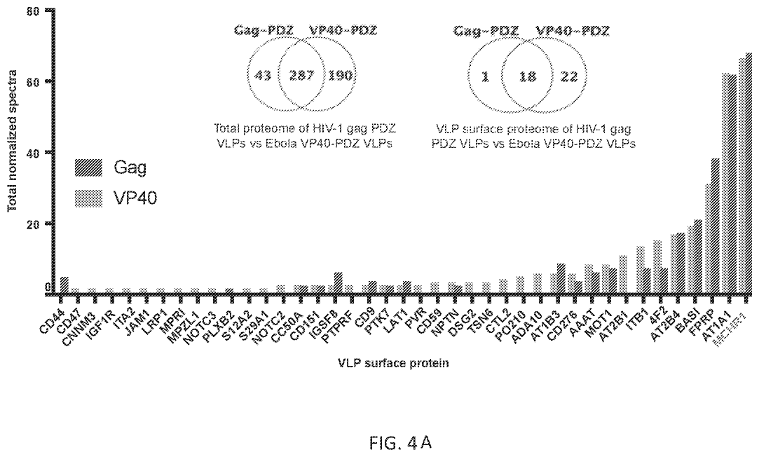

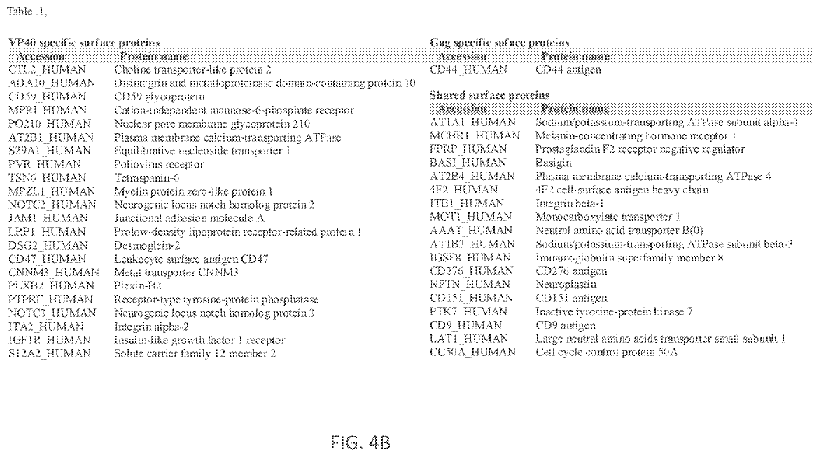

In order to establish the composition of the total proteomic and surfaceome composition of the Gag and Vp40 derived VLPs expressed in the Expi HEK293 expression system we performed LC-MS/MS analysis of PDZ-VLPs overexpressing MCHR1. We surprisingly found, as is demonstrated in FIG. 4 that the total proteome composition between HIV and Ebola derived VLPs differs significantly with Ebola VP40 capturing 143 additional host proteins compared to HIV-1. This could be explained with the relatively larger size of Ebola VP40 derived filamentous viral particles that are approximately 80-100 nm in diameter and of several μm in length enabling to recruit a larger host plasma membrane surface as an envelope and thus embedding more host proteins. As understanding the composition of the surfaceome of VLPs is of key interest in antibody discovery based assays and potentially vaccine development we performed a cross-reference analysis of the obtained VLP proteome with a list of the human surfaceome proteins (26). From this analysis we were able to characterise the obtained VLP surfaceome and to demonstrate that only a single cell surface protein CD44 was exclusive for HIV-1 and the remaining 18 host surface proteins derived from HIV-1 VLPs were shared with Ebola VP40 derived VLPs that contained a total of 40 cell surface proteins. As seen from FIG. 4 the most abundant detected cell surface protein based on observed peptide spectra is MCHR1 that was overexpressed to be captured on the VLPs. The complete list of cell surface proteins is presented in Table. 1 (FIG. 4B)

### Development of Wnt Signalling Modulating Antibodies by VLPs

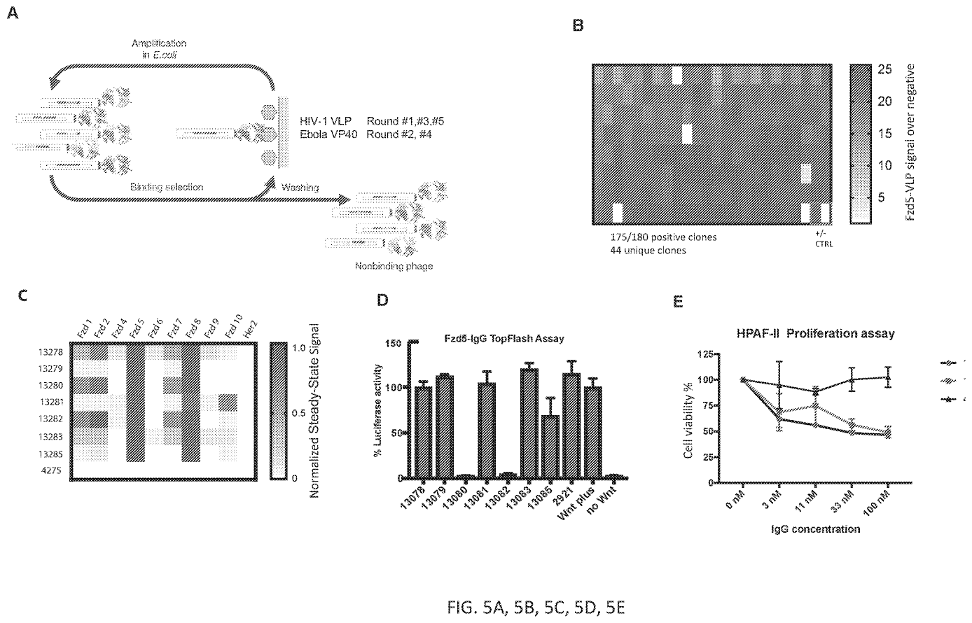

In order to demonstrate the ability to develop antibodies by using PDZ-VLPs we expressed a Class F GPCR Frizzled-5 on HIV-1 gag-PDZ and Ebola VP40-PDZ VLPs and used them as antigens for synthetic antibody discovery by phage display. The VLPs were coated on solid surface MaxiSorp immunoassay plates at 20 μg/ml and introduced to a synthetic Fab displaying antibody library F (27). The unbound phage were washed off and the bound phage was amplified overnight in Omnimax E. coli cells. A total of 5 rounds of selections was performed with alternating in each round between HIV-1 and Ebola derived Fzd5-VLPs as demonstrated in FIG. 5A. Each selection round additionally included a negative selection step where the phage antibody library was exposed to Fzd5 negative VLPs to remove any background binding phage. A total of 180 individual clones were analysed for specific binding to Fzd5-VLPs compared to negative-VLPs. As shown in FIG. 5B a total of 175 clones were positive in binding to Fzd5-VLPs with a Fzd5 signal over negative ratio over 10. Sequencing of all obtained phage clones identified a total of 44 unique antibody clones based on CDRH3 sequences. 7 unique antibody clones not obtained by previous antibody selection campaigns with recombinant Fzd5 extracellular CRD domain were taken to further characterization. As seen from a bio-layer interferometry assay in FIG. 5C the obtained antibody clones are all positive in binding Fzd5-CRD domain but have additional cross reactive binding specificity to other Frizzled isoforms. All obtained antibody clones were confirmed to be cross reactive to Frizzled 8 in addition to variable cross reactivity towards other Fzd isoforms throughout different clones. An antibody 4275 recognizing Gaussia-luciferase was used as a negative control.

To further demonstrate the potential ability of the obtained antibodies to modulate Wnt-Frizzled signalling we used the TopFlash assay in HEK293 cells to monitor beta-catenin translocation driven activation of TCF/LEF transcription factor activated luciferase expression upon Frizzled 5 binding to Wnt3a (28). Frizzled 5 was transiently overexpressed in HEK293 cells and 24 h post-transfection the cells were treated with Wnt3a and anti-Fzd5 antibodies. As demonstrated in FIG. 5D antibodies 13080 and 13082 were effective in blocking Wnt3a driven activation of luciferase indicating that these antibodies are acting as antagonists of Fzd5 driven canonical signalling.

As Fzd5 has been demonstrated to be essential for the proliferation of RNF43 mutant pancreatic cancer cells we tested the ability of 13080 and 13082 to inhibit the proliferation of HPAF-II pancreatic cancer cells (29). HPAF-II cells were treated with 0-100 nM concentration of 13080 and 13082 antibodies for 6 days and cell proliferation was analysed by Alamar-Blue assay. As seen in FIG. 5E HPAF-II cells are responsive to the Fzd5 antagonist antibodies with a dose dependent decrease of cell proliferation in response to antagonistic Fzd5 antibodies whereas the 4275 control IgG does not have an effect on cell viability. This demonstrates that HIV-1 and Ebola PDZ-VLPs can be utilized for synthetic antibody discovery by phage display and we were successful in obtaining functional Wnt-Fzd signalling modulating antibodies.

### LITERATURE

1. Oprea T I, Bologa C G, Brunak S, Campbell A, Gan G N, Gaulton A, et al. Unexplored therapeutic opportunities in the human genome. Nature Reviews Drug Discovery. 2018 May; 17 (5): 317-32.
2. Liu J K H. The history of monoclonal antibody development-Progress, remaining challenges and future innovations. Annals of Medicine and Surgery. 2014 December; 3 (4): 113-6.
3. Frenzel A, Schirrmann T, Hust M. Phage display-derived human antibodies in clinical development and therapy. mAbs. 2016 Oct. 2; 8 (7): 1177-94.
4. Bazan J, Całkosiński I, Gamian A. Phage display-A powerful technique for immunotherapy: 1. Introduction and potential of therapeutic applications. Human Vaccines & Immunotherapeutics. 2012 December; 8 (12): 1817-28.
5. Hutchings C J, Koglin M, Olson W C, Marshall F H. Opportunities for therapeutic antibodies directed at G-protein-coupled receptors. Nat Rev Drug Discov. 2017 September; 16 (9): 787-810.
6. Hutchings C J, Colussi P, Clark T G. Ion channels as therapeutic antibody targets. mAbs. 2019 Feb. 17; 11 (2): 265-96.
7. Sriram K, Insel P A. G Protein-Coupled Receptors as Targets for Approved Drugs: How Many Targets and How Many Drugs? Molecular Pharmacology. 2018 April; 93 (4): 251-8.
8. Jo M, Jung ST. Engineering therapeutic antibodies targeting G-protein-coupled receptors. Experimental & Molecular Medicine. 2016 February; 48 (2): e207-e207.
9. Frenzel A, Schirrmann T, Hust M. Phage display-derived human antibodies in clinical development and therapy. MAbs. 2016 Jul. 14; 8 (7): 1177-94.
10. Huang R, Kiss M, Batonick M, Weiner M, Kay B. Generating Recombinant Antibodies to Membrane Proteins through Phage Display. Antibodies. 2016 May 2; 5 (2): 11.
11. Kurg R, Reinsalu O, Jagur S, Ounap K, Vosa L, Kasvandik S, et al. Biochemical and proteomic characterization of retrovirus Gag based microparticles carrying melanoma antigens. Scientific Reports [Internet]. 2016 September [cited 2019 Dec. 10]; 6 (1). Available from: http://www.nature.com/articles/srep29425
12. Zeltins A. Construction and Characterization of Virus-Like Particles: A Review. Molecular Biotechnology. 2013 January; 53 (1): 92-107.
13. Tucker D F, Sullivan J T, Mattia K-A, Fisher C R, Barnes T, Mabila M N, et al. Isolation of state-dependent monoclonal antibodies against the 12-transmembrane domain glucose transporter 4 using virus-like particles. Proceedings of the National Academy of Sciences. 2018 May 29; 115 (22): E4990-9.
14. Cosson P. Direct interaction between the envelope and matrix proteins of HIV-1. The EMBO Journal. 1996 November; 15 (21): 5783-8.
15. Manrique J M, Affranchino J L, González S A. In vitro binding of simian immunodeficiency virus matrix protein to the cytoplasmic domain of the envelope glycoprotein. Virology. 2008 May; 374 (2): 273-9.
16. Laura R P, Witt A S, Held H A, Gerstner R, Deshayes K, Koehler M F T, et al. The Erbin PDZ Domain Binds with High Affinity and Specificity to the Carboxyl Termini of 8-Catenin and ARVCF. Journal of Biological Chemistry. 2002 Apr. 12; 277 (15): 12906-14.
17. Romero G, von Zastrow M, Friedman P A. Role of PDZ Proteins in Regulating Trafficking, Signaling, and Function of GPCRs: Means, Motif, and Opportunity. In: Advances in Pharmacology [Internet]. Elsevier; 2011 [cited 2019 Dec. 10]. p. 279-314. Available from: https://linkinghub.elsevier.com/retrieve/pil/B9780123859525000038
18. Gage R M, Matveeva E A, Whiteheart S W, von Zastrow M. Type I PDZ Ligands Are Sufficient to Promote Rapid Recycling of G Protein-coupled Receptors Independent of Binding to N-Ethylmaleimide-sensitive Factor. Journal of Biological Chemistry. 2005 Feb. 4; 280 (5): 3305-13.
19. Tao Y, Dai P, Liu Y, Marchetto S, Xiong W-C, Borg J-P, et al. Erbin regulates NRG1 signaling and myelination.: 6.
20. Gheysen D, Jacobs E, de Foresta F, Thiriart C, Francotte M, Thines D, et al. Assembly and release of HIV-1 precursor Pr55gag virus-like particles from recombinant baculovirus-infected insect cells. Cell. 1989 October; 59 (1): 103-12.
21. Noda T, Sagara H, Suzuki E, Takada A, Kida H, Kawaoka Y. Ebola Virus VP40 Drives the Formation of Virus-Like Filamentous Particles Along with GP. Journal of Virology. 2002 May 15; 76 (10): 4855-65.
22. Cervera L, Gutiérrez-Granados S, Martínez M, Blanco J, Gòdia F, Segura MM. Generation of HIV-1 Gag VLPs by transient transfection of HEK 293 suspension cell cultures using an optimized animal-derived component free medium. Journal of Biotechnology. 2013 July; 166 (4): 152-65.
23. Schülein R, Westendorf C, Krause G, Rosenthal W. Functional significance of cleavable signal peptides of G protein-coupled receptors. European Journal of Cell Biology. 2012 April; 91 (4): 294-9.
24. Spurgers K B, Alefantis T, Peyser B D, Ruthel G T, Bergeron A A, Costantino J A, et al. Identification of Essential Filovirion-associated Host Factors by Serial Proteomic Analysis and RNAi Screen. Molecular & Cellular Proteomics. 2010 December; 9 (12): 2690-703.
25. Santos S, Obukhov Y, Nekhai S, Bukrinsky M, Jordanskiy S. Virus-producing cells determine the host protein profiles of HIV-1 virion cores. Retrovirology. 2012; 9 (1): 65.
26. Bausch-Fluck D, Goldmann U, Müller S, van Oostrum M, Müller M, Schubert O T, et al. The in silico human surfaceome. Proceedings of the National Academy of Sciences. 2018 Nov. 13; 115 (46): E10988-97.
27. Persson H, Ye W, Wernimont A, Adams J J, Koide A, Koide S, et al. CDR-H3 diversity is not required for antigen recognition by synthetic antibodies. J Mol Biol. 2013 Feb. 22; 425 (4): 803-11.
28. Korinek V. Constitutive Transcriptional Activation by a beta-Catenin-Tcf Complex in APC−/− Colon Carcinoma. Science. 1997 Mar. 21; 275 (5307): 1784-7.
29. Genome-wide CRISPR screens reveal a Wnt-FZD5 signaling circuit as a druggable vulnerability of RNF43-mutant pancreatic tumors| Nature Medicine [Internet]. [cited 2018 Dec. 11]. Available from: https://www.nature.com/articles/nm.4219

What is claimed is:

1. A virus-like-particle (VLP) expressing one or more membrane embedded proteins on its surface and comprising a viral scaffolding protein fused to a PDZ domain and a synthetic polypeptide in cytoplasmic C-terminal tail of the one or more target membrane proteins, and optionally a signal peptide incorporated at the N-terminal end of the one or more target membrane proteins.

2. The VLP of claim 1, wherein the signal peptide is a signal peptide of Albumin, Melittin, Pro-MCH, Gaussia luciferase, Heamagluttinin, HIV Glycoprotein, LRRCP32, Growth Hormone Receptor, Proteinase activated Receptor, Tissue type Plasminogen activator, Secrecon, AcMNPV, Somatrotopin, IL-2, or MCHR-1, but not limited to these.

3. The VLP of claim 1, wherein the PDZ domain is according to SEQ ID NO:3.

4. The VLP of claim 1, wherein the viral scaffolding protein is Ebola virus VP40 or HIV-1 virus gag.

5. The VLP of claim 1, wherein the one or more membrane protein is selected from the group consisting of G-protein coupled receptors (GPCRs), ion channels, tetraspanins and single-pass membrane proteins.

6. The VLP according to claim 1, wherein the synthetic polypeptide fused to the pseudotyped membrane protein is according to SEQ ID NO:6.

7. A VLP-vaccine or a VLP-preparation consisting of or comprising the VLP claim 1.

8. The VLP vaccine or VLP-preparation of claim 7, wherein the vaccine or preparation is an injectable, an intranasal, or an inhalable vaccine.

9. A method to generate an immune response, the method comprising administering to an animal a vaccine or preparation comprising or consisting of the VLP of claim 1.

10. The method of claim 9, wherein the method further comprises obtaining antibodies from VLP immunized animals or using VLPs of claim 1 in antibody screening and discovery process.

11. A method to develop monoclonal antibodies in a phage display system by using the VLPs according to claim 1.

12. An antibody obtained by the method of claim 10.

13. An antibody according to claim 12 for treatment of a disease.

14. A membrane protein expression system comprising virus-like particles according to claim 1, wherein the expression system enables expression and capturing of the membrane proteins on VLP surface in levels that are enhanced as compared to a system comprising wild-type VLP construct.

15. The VLP of claim 2, wherein the viral scaffolding protein is Ebola virus VP40 or HIV-1 virus gag.

16. The VLP of claim 3, wherein the viral scaffolding protein is Ebola virus VP40 or HIV-1 virus gag.

17. An antibody obtained by the method of claim 11.

18. An antibody according to claim 17 for treatment of a disease.


SEQUENCE LISTING (6 sequences)

SEQ ID NO: 1 - protein, 438 aa, synthetic construct
MGSHMGHELAKQEIRVRVEKDPELGFSISGGVGGRGNPFRPDDDGIFVTRVQPEGPASKLLQPGDKIIQANGYSFINIEHGQAVSLLKTFQNTVELIIVREVSSGSGGGSGGTRRVILPTAPPEYMEAIYPARSNSTIARGGNSNTGFLTPESVNGDTPSNPLRPIADDTIDHASHTPGSVSSAFILEAMVNVISGPKVLMKQIPIWLPLGVADQKTYSFDSTTAAIMLASYTITHFGKATNPLVRVNRLGPGIPDHPLRLLRIGNQAFLQEFVLPPVQLPQYFTFDLTALKLITQPLPAATWTDDTPTGSNGALRPGISFHPKLRPILLPNKSGKKGNSADLTSPEKIQAIMTSLQDFKIVPIDPTKNIMGIEVPETLVHKLTGKKVTSKNGQPIIPVLLPKYIGLDPVAPGDLTMVITQDCDTCHSPASLPAVVEK

SEQ ID NO: 2 - protein, 603 aa, synthetic construct
MGARASVLSGGELDKWEKIRLRPGGKKKYQLKHIVWASRELERFAVNPGLLETSEGCRQIMGQLQPSLQTGSEELRSLYNTVATLYCVHQKIEVKDTKEALDKVEEEQNNSKKKAQQEAADAGNRNQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIKQGPKEPFRDYVDRFYKTLRAEQATQEVKNWMTETLLVQNANPDCKTILKALGPAATLEEMMTACQGVGGPGHKARVLAEAMSQVTGSAAIMMQRGNFRNQRKTVKCFNCGKEGHIARNCRAPRKKGCWKCGKEGHQMKDCTERQANFLGKIWPSHKGRPGNFLQSRPEPTAPPEESFRFGEATAPSQKQEPIDKELYPLASLKSLFGSDPSSQGGGGSGHELAKQEIRVRVEKDPELGFSISGGVGGRGNPFRPDDDGIFVTRVQPEGPASKLLQPGDKIIQANGYSFINIEHGQAVSLLKTFQNTVELIIVREVSS

SEQ ID NO: 3 - protein, 98 aa, synthetic construct
HELAKQEIRVRVEKDPELGFSISGGVGGRGNPFRPDDDGIFVTRVQPEGPASKLLQPGDKIIQANGYSFINIEHGQAVSLLKTFQNTVELIIVREVSS

SEQ ID NO: 4 - protein, 441 aa, synthetic construct
MDYKDDDDKMSVGAMKKGVGRAVGLGGGSGCQATEEDPLPDCGACAPGQGGRRWRLPQPAWVEGSSARLWEQATGTGWMDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGTGSGTGWETWV

SEQ ID NO: 5 - protein, 458 aa, synthetic construct
MKWVTFISLLFLFSSAYSDYKDDDDKMSVGAMKKGVGRAVGLGGGSGCQATEEDPLPDCGACAPGQGGRRWRLPQPAWVEGSSARLWEQATGTGWMDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGTGSGTGWETWV

SEQ ID NO: 6 - protein, 7 aa, synthetic construct
TGWETWV